(19) United States
(12) Patent Application Publication
(10) Pub. No.: US 2024/0123209 A1
(43) Pub. Date: Apr. 18, 2024

(54) ADAPTOR FOR INFUSION LINE IDENTIFICATION SYSTEM

(71) Applicant: ORION INNOVATIONS INC., St. George, UT (US)

(72) Inventors: Wayne A. Provost, St. George, UT (US); Rodney W. Schutt, St. George, UT (US); Taylor Charles Hill, St. George, UT (US); Jonathan Neil Hart, St. George, UT (US)

(21) Appl. No.: 18/381,563

(22) Filed: Oct. 18, 2023

Related U.S. Application Data
(60) Provisional application No. 63/417,281, filed on Oct. 18, 2022.

(51) Int. Cl. A61M 39/00

(52) U.S. Cl. CPC A61M 39/00; A61M 2039/1077

(57) ABSTRACT

Disclosed are adaptor devices configured for attaching an indicator, such as a light source, to an infusion line to aid a user in identifying the infusion line, matching different sections of an infusion line as belonging to the same infusion line, distinguishing the infusion line from other infusion lines, and/or sensing of various parameters of the medical line. The adaptor device includes a receiving surface and one or more sets of laterally opposing grip elements projecting from the receiving surface. The grip elements are configured to accommodate medical lines of variable size.

### CROSS-REFERENCE TO RELATED APPLICATIONS

This application claims priority to and the benefit of U.S. Provisional Patent Application No. 63/417,281, filed Oct. 18, 2022 and titled “ADAPTOR FOR INFUSION LINE IDENTIFICATION SYSTEM,” the entirety of which is incorporated herein by reference.

### BACKGROUND

### Technical Field

This disclosure relates to devices configured for attachment to a medical line such as an infusion line to enable identification of the medical line and/or sensing of various parameters of the medical line.

### Related Technology

In certain medical settings such as hospital settings, patients are often administered therapeutic fluids via infusion lines, also typically referred to as intravenous (IV) lines. Infusion lines generally consist of flexible, polymer tubing connected at one end to a fluid source and at another end to a needle or port that provides access to a vessel of a patient. It is not uncommon for multiple infusion lines, each connected to a different source of fluid, to be used simultaneously to deliver several therapeutic fluids at once to a single patient. It is also not uncommon for the needles or ports to be located adjacent to one another, such as multiple adjacent needles providing access into the brachial vein running through the arm of the patient.

While the simultaneous use of multiple infusion lines can provide numerous benefits, challenges can also be encountered. For instance, when multiple infusion lines are used to administer multiple therapeutic fluids to a single patient, it can become cumbersome and difficult to readily identify one line from another. Thus, it can be difficult to quickly and accurately identify a particular therapeutic fluid source and corresponding therapeutic fluid output compared to another medication source and its corresponding therapeutic fluid output. This problem is aggravated by the tendency of each of the lines to coil up to their packaged configuration and consequently tangle with other lines or tangle under bed sheets or clothing.

Quick identification of a particular therapeutic fluid source is often required in emergency situations. For example, when a patient hooked up to multiple lines is in need of emergency intravenous administration of a therapeutic fluid not currently being provided through one of the lines, it is necessary to immediately provide that therapeutic fluid. If a blood vessel cannot rapidly be located into which the therapeutic fluid can be injected, it is common practice to provide the drug through an infusion line in which a therapeutic fluid is already being administered. This practice of using existing infusion lines to administer new therapeutic fluids is also common in non-emergency situations.

The person administering the drug, must be sure that the infusion line through which the new therapeutic fluid is administered is carrying a therapeutic fluid which is compatible with the new therapeutic fluid. Severe results may occur if a new therapeutic fluid is injected through an infusion line in which the therapeutic fluid already flowing therethrough is not compatible with the new therapeutic fluid. For example, if heparin is injected into an infusion line through which lidocaine is already flowing, a flakey precipitate will form in the mixture which can be dangerous to a patient. Similarly, mixing insulin with certain chemotherapy drugs in a common infusion line can be extremely dangerous for a patient.

Further complicating this issue is the fact that the medical field uses multiple different line diameters. Different line diameters are often used depending on clinical setting, equipment utilized, patient demographics (e.g., neonate, child, adult), and other particular application needs. For example, infusion pumps will often use relatively larger diameter tubing, while syringe pumps tend to use smaller tubing. In some instances, there may be lines of various sizes simultaneously connected to the same patient. As a result, even when caretakers seek to utilize indicator devices to help identify and distinguish different lines, the indicator devices may not attach properly, may inadvertently become detached, or are excessively difficult to properly attach in the first place.

As a result of the difficulties in distinguishing between multiple infusion lines and their associated fluid sources and outputs and the potentially life-threatening possibilities that can occur if incompatible therapeutic fluids are injected through the same infusion line, there is a need for devices that allow for ready and accurate identification of individual infusion lines with their associated fluid sources and outputs.

### SUMMARY

Described herein are adaptors beneficially configured to enable connection of indicator devices to infusion lines, and/or other medical lines, of varying diameters. The described adaptors beneficially enable users to attach indicator devices to a wide variety of infusion lines. This increases the versatility and functional flexibility of indicator devices and/or sensor devices that can be connected to infusion lines for purposes of identifying/distinguishing infusion lines and/or sensing various parameters of the infusion lines.

Note that although the specific examples discussed herein relate to infusion lines, the skilled person will readily understand that the same components and principles may be applied to adaptors for other types of medical lines where sensing and/or identification may be beneficial. For example, embodiments described herein may be connected to other types of intravenous (IV) lines such as central lines (e.g., central venous catheter lines) and peripherally inserted central catheter (PICC) lines, nasogastric tubes, arterial lines, peritoneal dialysis lines, umbilical lines (typically used for newborns), long-term access catheters (e.g., Hickman or Broviac catheters), wound drainage lines, or any other medical line where integration with sensor and/or indicator functionality is beneficial.

The adaptor devices disclosed herein include features enabling attachment to infusion lines of varying diameters. The adaptor devices can also be configured to house one or more indicator components (e.g., lights, speakers, etc.), one or more sensors (e.g., sensors for determining air bubbles, flow rate, precipitate formation, temperature, salinity, etc.), and/or one or more communications components (e.g., wireless transmitters, wireless receivers) for communicating with other indicator devices and/or external computer devices. The indicator component(s) can activate based on sensor activity (e.g., upon detection of an alarm condition), to signal various status conditions (e.g., normal, warning, emergency), or according to manual activation (e.g., simultaneously activating other connected indicator devices associated with the same infusion line).

The adaptor devices disclosed herein can beneficially provide ease of use and reduced risk of clinician error. The adaptor devices are also able to be effectively secured to a target infusion line, with minimal risk of subsequent sliding or disconnection, and without significantly affecting fluid flow even for different line diameters.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a conventional infusion line assembly; and

FIGS. 2A and 2B illustrate an example adaptor device configured to incorporate an indicator and configured to enable attachment to a variety of sizes of infusion lines, the adaptor device including sets of grip elements wherein each set includes a stationary grip element opposite a biased grip element;

FIGS. 3A and 3B illustrate portions of example adaptor devices comprising grip elements that include fins capable of biasing toward an infusion line held thereby;

FIG. 4 illustrates a portion of an example adaptor device including sets of curved grip elements that are biased toward one another to secure an infusion line;

FIG. 5 illustrates a portion of an example adaptor device wherein each set of grip elements includes a stationary grip element and an opposed curved grip element;

FIGS. 6A and 6B illustrate portions of example adaptor devices comprising grip elements arranged with a rotational and/or longitudinal offset;

FIG. 7 illustrates a portion of an example adaptor device comprising grip elements that include angled sections to help force a bend in a received infusion line;

FIG. 8 illustrates a portion of an example adaptor device comprising a set of three or more laterally coincident grip elements arranged to define two or more infusion line paths of different size; and

FIGS. 9A and 9B illustrate portions of example adaptor devices wherein grip elements are configured as guide posts for forcing a bend in an infusion line to help secure the infusion line.

### DETAILED DESCRIPTION

### Introduction

The present disclosure relates to devices and methods for identification of infusion lines. Such are configured to enable the reliable identification of one infusion line from another in a simple and efficient manner. Such embodiments can beneficially function to prevent the inadvertent injection of incompatible therapeutic fluids through a single infusion line. An infusion line identification device as described herein may reduce the number of misidentified infusion lines without significant changes to the existing clinical methods and/or equipment.

The presently described embodiments may be practiced using infusion line systems and related devices and methods and general manufacturing techniques known in the art. Such details are known to the skilled person and are not described in detail.

While infusion line therapy devices for administering therapeutic fluids are exemplified below, other embodiments may be implemented in other applications, both within the medical field and in other technical fields. Various components of the illustrative embodiments may be excluded or replaced with other components known and used in the art. By way of non-limiting example, some of the exemplary embodiments include therapeutic fluid bags, pumps, and connectors. Each of these components could be eliminated or replaced with other components. For instance, various types of pumps, or no pump at all, can be used with the disclosed embodiments. Similarly, various types of fluid sources and connectors other than conventional therapeutic fluid bags and Y-connectors could be employed.

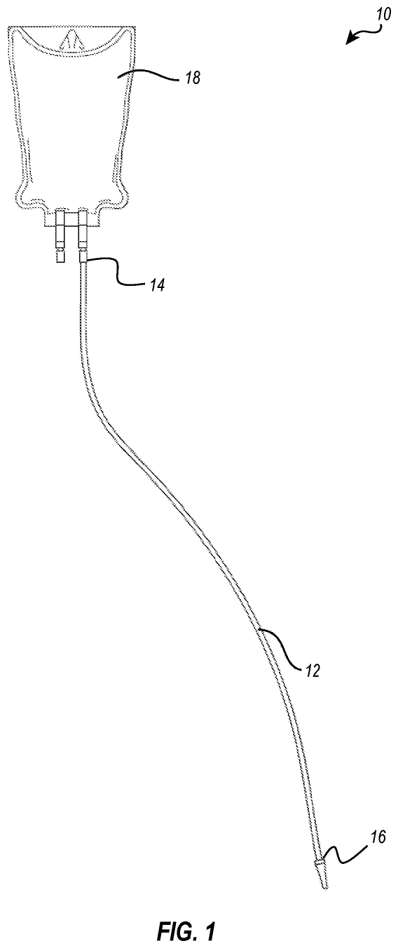

FIG. 1 illustrates a conventional infusion line assembly 10 including an infusion line 12 connected to a therapeutic fluid bag 18. The infusion line 12 includes a first end 14 and a second end 16. As shown, one end may be connected to the therapeutic fluid bag 18, leaving the other end free to be intravenously inserted into the patient. As discussed above, issues can arise in medical settings due to the difficulty in properly matching one end of an infusion line to its opposite end. That is, where multiple infusion lines are associated with a patient, it can often be difficult to readily identify which line at the patient end is attached to which therapeutic fluid.

U.S. Pat. No. 10,722,641, which is incorporated herein in its entirety by this reference, describes infusion line assemblies configured to enable identification of an infusion line via multiple light sources that are selectively attachable to an infusion line and that are communicatively coupled to one another via a wireless connection. The light sources are configured such that when a first light source is manually activated to provide a light signal, a second light source is also automatically activated, without requiring manual activation, to provide a corresponding light signal, and vice versa.

Such selectively attachable indicator devices beneficially enable users (e.g., medical personnel) to easily attach the devices to an infusion line, or multiple sets of such indicator devices to multiple respective infusion lines, in a conventional setting without the need for additional attachment equipment or custom manufacturing processes. However, infusion lines do not have a uniform size, and thus such indicator devices may not readily attach to all types of infusion lines or may attach better to some types of infusion lines than others.

Described herein are adaptors beneficially configured to enable connection of indicator devices to infusion lines of varying diameters. The described adaptors beneficially enable users to attach indicator devices such as those disclosed in U.S. Pat. No. 10,722,641 to be readily attached to a wide variety of infusion lines. This increases the versatility and functional flexibility of indicator devices for identifying and/or distinguishing infusion lines.

### Adaptor Devices

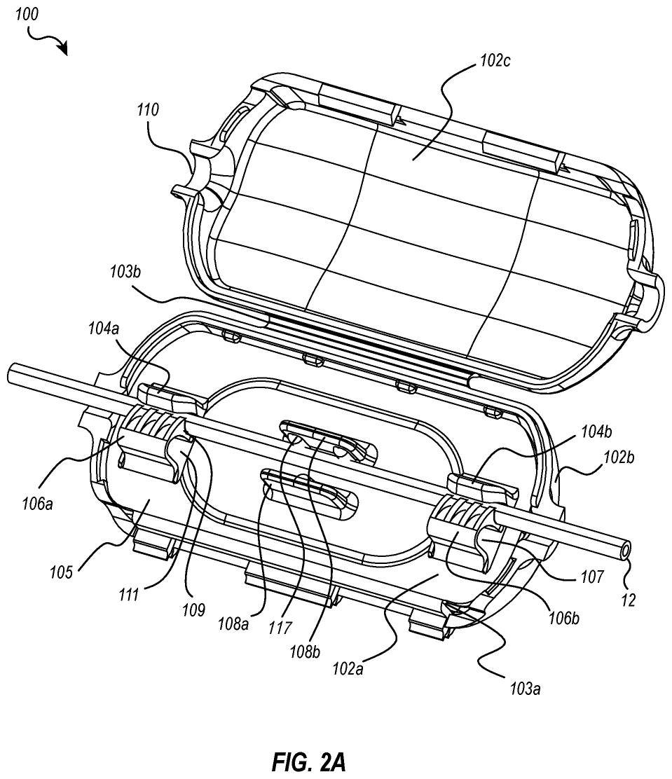

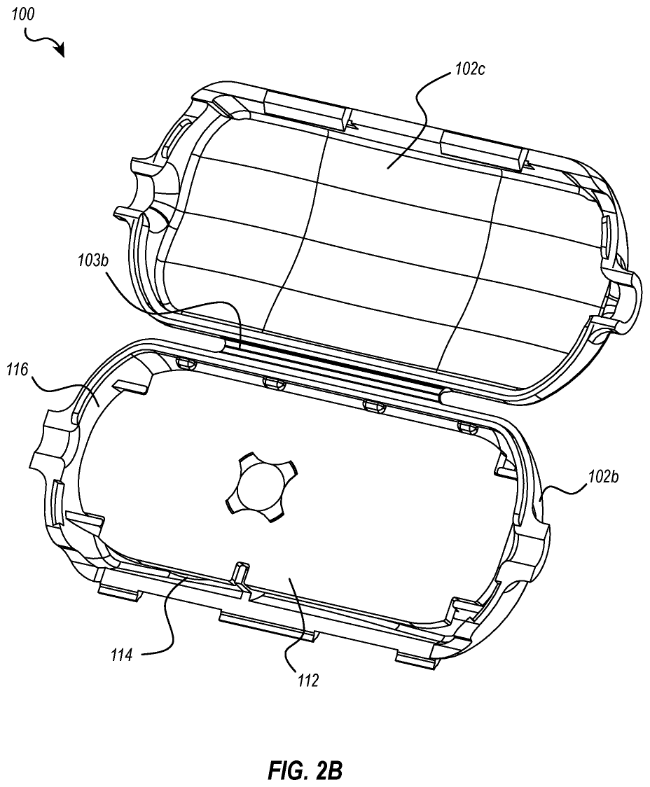

FIGS. 2A and 2B illustrate an example adaptor device 100 configured to incorporate an indicator (e.g., a light source) and configured to enable attachment to a variety of sizes of infusion lines. An infusion line 12 is also shown for purposes of illustration.

FIG. 2A is an isometric view of the adaptor device 100. As shown, the adaptor device 100 includes three sections 102a, 102b, and 102c arranged in a trifold configuration. That is, section 102a can be folded with respect to section 102b along hinge 103a, and section 102c can be folded with respect to section 102b along hinge 103b. Connectors may be disposed at or near hinge 103a and/or 103b to selectively fix the respective sections in the closed position. Such connectors may include, for example, flexible tabs, friction fittings, magnetic attachments, or other suitable fixing means. When the sections 102a, 102b, and 102c are placed in the closed position, the device 100 can function as a housing that includes apertures 110 through which the infusion line 12 can pass.

In the illustrated embodiment, hinges 103a and 103b are disposed on opposite sides of the section 102b. In other embodiments, hinges 103a and 103b are disposed on the same side. In yet other embodiments, hinge 103a and/or hinge 103b are omitted. For example, the separate sections 102a, 102b, and 102c may be selectively assembled to form the closed configuration of the adaptor device 100 without being connected via hinges 103a and/or 103b.

In some embodiments, two or more of sections 102a, 102b, and 102c may be integrally formed as one piece, without an associated hinge. For example, although constructing sections 102a and 102b to be rotatable and/or detachable relative to one another enables access to an inner compartment 116 (see FIG. 2B) for housing the indicator, power source, and/or other components, other embodiments may enclose the inner compartment 116 during manufacture.

In some embodiments, one or more of sections 102a, 102b, or 102c may be omitted. For example, although section 102c is preferred in order to form a complete enclosure around the corresponding section of the infusion line 12, it may be omitted in some embodiments.

As shown in the illustrated embodiment, when section 102a is folded into a closed position with respect to section 102b, a receiving surface 105 of section 102a is positioned to receive the infusion line 12. Grip elements 104 (including 104a and 104b) and opposing grip elements 106 (including 106a and 106b) project from the receiving surface 105 and are configured to grip the infusion line 12 to enable attachment of the adaptor device 100 to the infusion line 12.

In the illustrated adaptor device 100, the grip elements 104 are configured as stationary grip elements, whereas the grip elements 106 are configured as biased grip elements. The biased grip elements 106 include features that bias the grip surface 109 of the grip element (i.e., the surface that contacts the infusion line 12) toward the opposing stationary grip element 104. This biasing feature enables the biased grip elements 106 to flex relative to the stationary grip elements 104 to accommodate a variety of sizes of infusion lines 12.

In other words, where a longitudinal axis is defined as the axis along which the infusion line 12 sits (i.e., from one aperture 110 to the other), the biased grip elements 106 are configured to flex along a lateral axis that runs substantially perpendicular to the longitudinal axis, to thereby vary the space between each biased grip element 106 and a corresponding stationary grip element 104.

The illustrated embodiment shows two sets of grip elements, each with a stationary grip element 104 and a biased grip element 106. Other embodiments may include different arrangements of grip elements. For example, some embodiments may include a single stationary grip element 104 and a single biased grip element 106. Other embodiments may include three or more sets of grip elements each with a stationary grip element 104 and a single biased grip element 106.

Moreover, although the illustrated embodiment shows a single stationary grip element 104 for every biased grip element 106 and vice versa, other embodiments may arrange the relationship differently. For example, some embodiments may include one or more sets with multiple stationary grip elements 104 to a single biased grip element 106 and/or one or more sets with multiple biased grip elements 106 to a single stationary grip element 104.

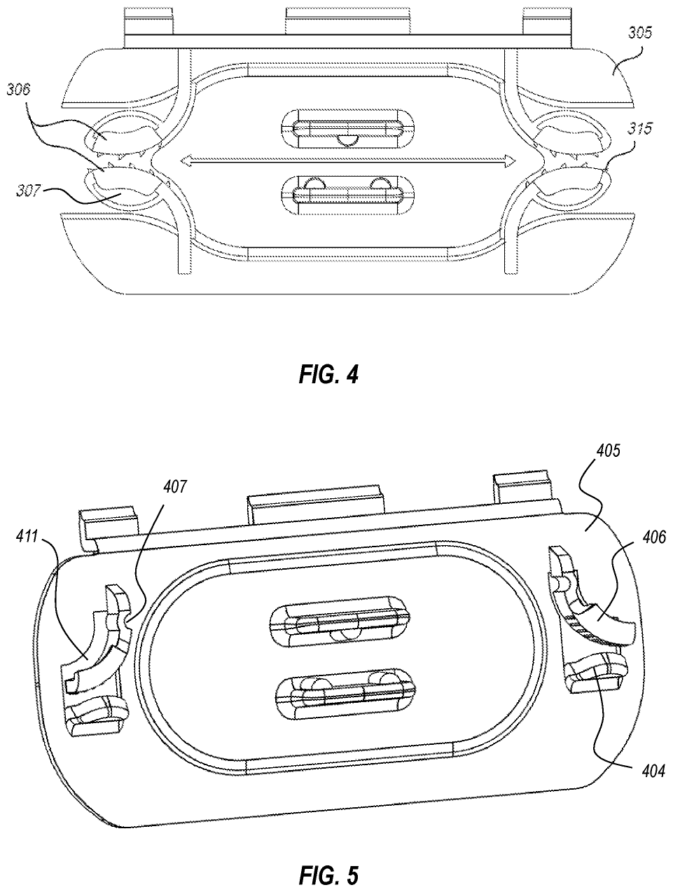

In the illustrated embodiment, the biased grip elements 106 include an arch structure with a variable span 107 that varies according to level of bias of the biased grip element 106. That is, the span 107 is greater when the grip surface 109 of the biased grip element 106 is more biased toward the corresponding stationary grip element 104, whereas the span 107 lessens when the grip surface 109 flexes away from the stationary grip element 104 (e.g., when pushed away by an infusion line inserted therebetween). Here, the arch structure that forms the variable span 107 sits on a vertical plane to allow expansion and contraction along a lateral direction. The arch structure may alternatively extend along a horizontal plane (see, e.g., the arch structure and variable span 307 of FIG. 4).

As shown, the span 107 of each biased grip element 106 is able to collapse and expand because section 102c includes corresponding channels 111 that allow the grip surfaces 109 to move laterally. That is, the grip surfaces 109 are not fixed to the receiving surface 105 and the channels 111 allow the grip surfaces 109 to move toward and away from their corresponding stationary grip elements 104.

FIG. 2B illustrates a view of the adaptor device 100 with section 102c removed to illustrate the inner compartment 116. In this embodiment, the inner compartment 116 includes a circuit board 112 and power source 114 (e.g., battery), which can function as an indicator (e.g., light source) for identifying an infusion line to which the adaptor device 100 is attached, optionally along with one or more additional adaptor devices configured to provide a similar indication (e.g., a similar color of light). In this manner, different sections of the same infusion line to which the multiple adaptor devices are attached can be readily identified as belonging to the same infusion line.

The adaptor device 100 may also include one or more centrally located grip elements 108, such as grip elements 108a and 108b, disposed at or near the longitudinal center of the device 100 (e.g., between the more peripheral grip elements 104, 106). Such elements may function to provide additional structure to the device, to provide additional protection to the infusion line 12 against kinking, impinging, or bending, and/or additional positional guidance of the infusion line 12 through the device.

The grip elements 108 may be formed as vertical projections extending from the receiving surface 105 to form structural guides and/or barriers for limiting unwanted movement of a received infusion line 12. Although a single pair of grip elements 108 is shown here, other embodiments may include multiple centrally located grip elements or may omit centrally located grip elements.

The centrally located grip elements 108 may also include one or more lateral projections 117 that extend toward the inner space intended for receiving the infusion line 12 to aid in securing or guiding the infusion line 12. These lateral projections 117 can be longitudinally offset from one another so as to support and abut a received infusion line without creating a potential pinch point in the line. Although not included in the illustrated examples, some embodiments may additionally or alternatively include lateral projections on the peripherally located grip elements 104, 106 with similar functionality.

An adaptor device as disclosed herein may include one or more sets of peripherally located grip elements (such as grip elements 104, 106), one or more sets of centrally located grip elements (such as grip elements 108), or both. Either or both types of grip elements may include biasing features to assist in securing to an infusion line.

Other embodiments may additionally or alternatively include other configurations of grip elements to enable attachment to variably sized infusion lines. The following embodiments can additionally or alternatively include any of the features discussed above with respect to adaptor device 100, and vice versa. For example, an embodiment may include any combination of grip elements disclosed herein, even if every combination is not specifically illustrated.

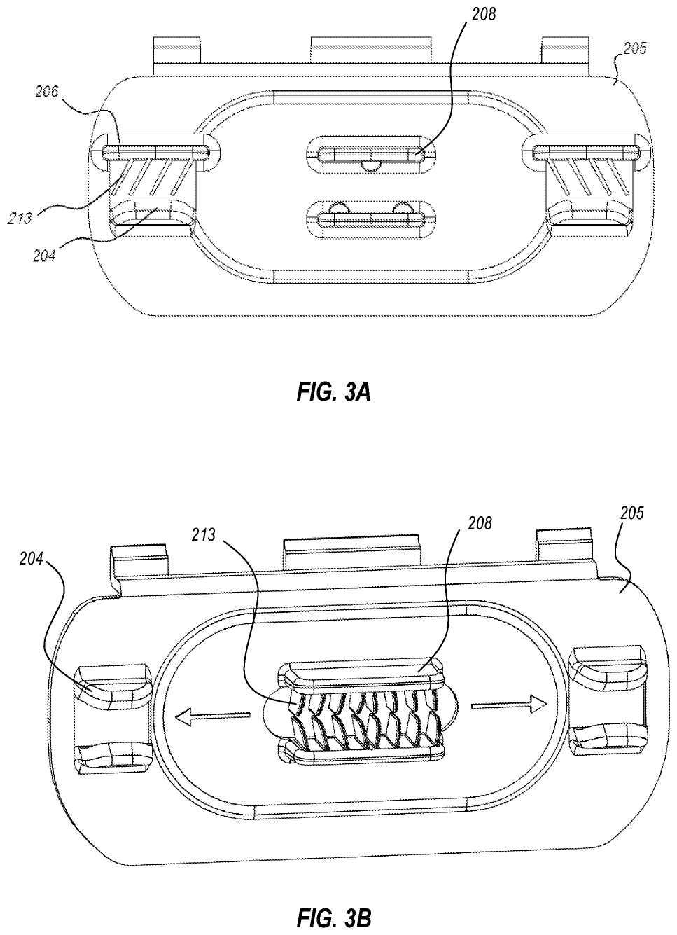

FIG. 3A shows a receiving surface 205 on which stationary grip elements 204 are disposed opposite biased grip elements 206 at peripheral locations of the device. The biased grip elements 206 include one or more fins 213 that bias toward the opposing stationary grip element 204 and are capable of rotating from a more lateral orientation to a more longitudinal orientation to provide more space between the opposing grip elements 204, 206 to thereby accommodate infusion lines of differing sizes while maintaining a biasing force against the line once positioned within the device. The illustrated embodiment also includes centrally located grip elements 208. In this embodiment, the centrally located grip elements 208 omit fins.

Although the illustrated embodiment shows that each set of grip elements includes a stationary grip element 204 opposite a biased grip element 206 with fins 213, alternative embodiments can include opposing grip elements that both include fins 213 extending toward one another.

FIG. 3B illustrates another configuration of receiving a surface 205 that utilizes fins 213. In this configuration, the fins 213 extend inwardly from the centrally located grip elements 208. As in FIG. 3A, the fins 213 can be moved to a more longitudinal orientation to make space to accommodate a received infusion line, while also biasing toward the more lateral position to thereby aid in securing the infusion line. The illustrated embodiment shows that both opposing grip elements 208 include fins 213. Alternatively, one grip element 208 may omit fins (similar to the grip elements 204 and 206 shown in FIG. 3A). The peripherally located grip elements 204 can omit fins (as shown) or can include additional fins.

FIG. 4 shows a receiving surface 305 that includes grip elements 306 that are each biased toward an opposing grip element 306. That is, for a given pair of grip elements 306, each of the opposing grip elements are biased toward one another. This is in contrast to a configuration (such as illustrated in FIG. 2A) that includes a stationary grip element and a biased grip element. The grip elements 306 are biased toward one another but are capable of rotating on the horizontal plane to expand the space between grip elements 306 to accommodate an infusion line.

As shown, grip elements 306 can include one or more projections 315 (e.g., ridges, teeth, grooves, etc.) to increase friction and hold against a received infusion line.

Similar to the embodiment shown in FIG. 2A, the grip elements 306 can include a structural feature that enables a variable span 307. That is, the span 307 can collapse to enable a grip element 306 to move away from an opposing grip element 306, and can expand to bias the grip element 306 toward the opposing grip element 306. Here, the arch structure forming the variable span 307 sits on a horizontal plane, whereas the arch structure forming the variable span 107 in FIG. 2A sits on a vertical plane. Biasing forces can additionally or alternatively be provided by the curved shape of the grip element 306 itself.

FIG. 5 shows another embodiment of a receiving surface 405. This embodiment includes stationary grip elements 404 and biased grip elements 406. In this embodiment, the biased grip elements 406 have a curved shape and are capable of rotating upon the horizontal plane, similar to the grip elements 306 of FIG. 4. However, in this embodiment, the grip elements 406 are disposed within corresponding channels 411 that allow the grip elements 406 to move on the horizontal plane.

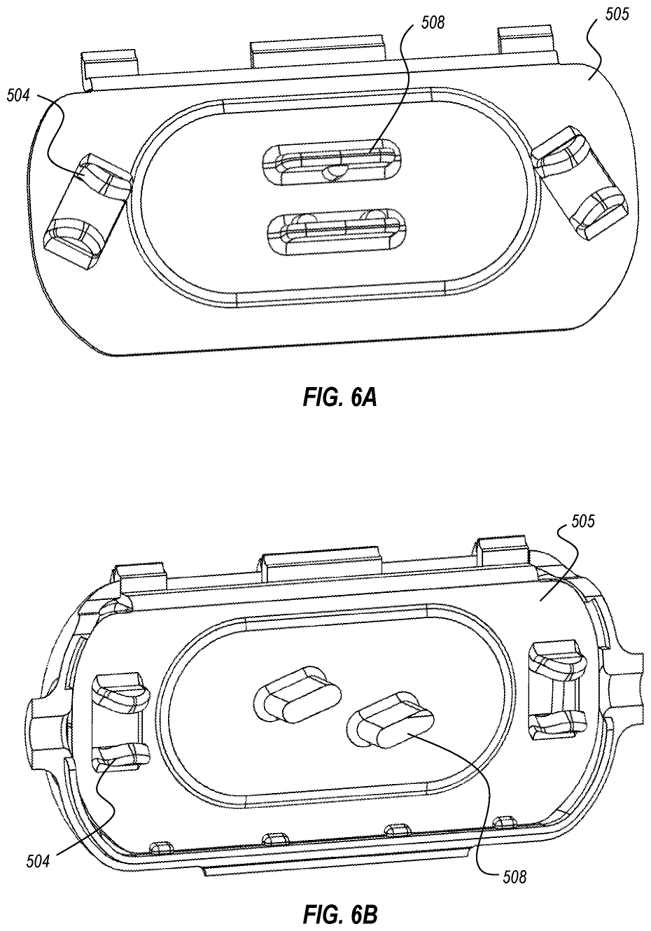

FIGS. 6A and 6B show other embodiments of receiving surfaces 505 with grip elements 504 and/or 508 arranged with a rotational offset and/or offset along the longitudinal direction. Such embodiments can beneficially induce a bend in a received infusion line that aids in securing the device to the infusion line, but without overly bending to cause kinking or impinging of the line.

In FIG. 6A, for example, the centrally located grip elements 508 are substantially parallel to the longitudinal axis of the device. However, each set of the peripherally located grip elements 504 are rotationally offset such that the widths of the grip elements 504 extend in a direction transverse to the longitudinal axis. This forces the infusion line into a bend shape and causes the infusion line to push against the walls of the grip elements 504 to aid in securing the device to the infusion line.

Each set of grip elements 504 can be independently rotated relative to the longitudinal axis an amount suitable to provide the desired bend in a received infusion line. For example, each set of grip elements 504 may be independently rotated relative to the longitudinal axis (in either direction) by about 10 degrees to about 45 degrees, or about 15 degrees to about 40 degrees, or about 20 degrees to about 35 degrees, or an amount within a range using any combination of the foregoing as endpoints. The sets of grip elements 504 may be rotated in opposite directions, as shown, or may be rotated in the same direction.

In FIG. 6B, the peripherally located grip elements 504 are substantially parallel to the longitudinal axis, and the centrally located grip elements 508 are rotated relative to the longitudinal axis. The centrally located grip elements 508 can also be slightly offset along the longitudinal direction, as shown. As with the sets of peripherally located grip elements 504 in FIG. 6A, the centrally located grip elements 508 of FIG. 6B force a gentle bend in a received infusion line to help secure the infusion line without overly bending to cause kinking or impingement.

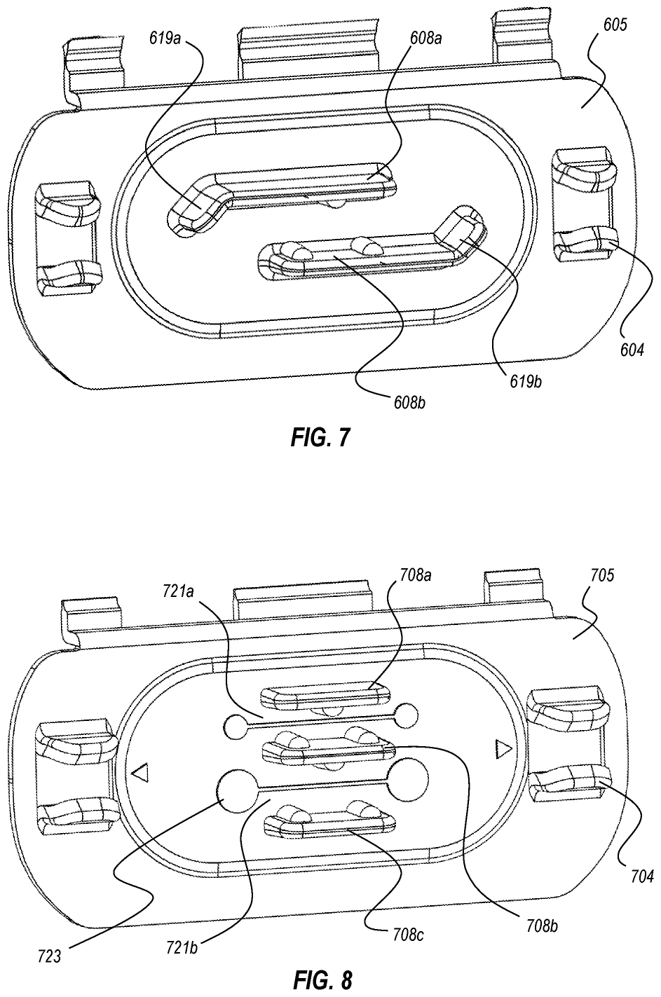

FIG. 7 illustrates another embodiment of a receiving surface 605 in which one or more grip elements include angled sections to help force a bend in a received infusion line. In the illustrated embodiment, each of the centrally located grip elements 608 includes a main section that is substantially parallel with the longitudinal axis and an angled section 619 that angles inward toward the center (i.e., toward the longitudinal axis). A first grip element 608a of a grip element pair includes a first angled section 619a on a first side (i.e., the “left” side from the view shown), and a second grip element 608b of the grip element pair includes a second angled section 619b on a second side opposite the first side (i.e., the “right” side from the view shown). The grip elements 608 therefore work together to define a curved path for an infusion line received therebetween.

The embodiment shown in FIG. 7 includes peripherally located grip elements 604 that are substantially parallel with the longitudinal axis of the device. One or more of such grip elements 604 may be angled (such as shown in FIG. 6A) in alternative embodiments.

FIG. 8 illustrates another embodiment of a receiving surface 705 in which the grip elements define multiple infusion line paths. The illustrated embodiment includes peripherally located grip elements 704 that help to define the infusion line path, and includes at least three centrally located grip elements 708 that define multiple infusion line paths. For example, a first infusion line path 721a can be disposed between a first (i.e., “top”) grip element 708a and a second (i.e., “middle”) grip element 708b, while a second infusion line path 721b can be disposed between the second grip element 708b and a third (i.e., “bottom”) grip element 708c. Additional grip elements can be laterally stacked to define even more infusion line paths.

As shown, the infusion line paths 721 need not be the same size. In some circumstances, it can be beneficial to provide infusion line paths 721 of different sizes to enable the user to select the infusion line path 721 that best matches a particular infusion line. In the illustrated embodiment, infusion line path 721a is sized to be relatively smaller, whereas infusion line path 721b is sized to be relatively larger. The user can select the path that best fits a particular infusion line without needing to resort to different adaptor devices. For example, an infusion line that risks excessive pinching when placed in smaller infusion line path 721a can instead be positioned in infusion line path 721b, whereas an infusion line that is too small to be properly secured within infusion line path 721b can instead be positioned in infusion line path 721a.

The receiving surface 705 can also include one or more indicator markings 723 to aid the user in identifying how to route the infusion line through the device. For example, the indicator markings 723 can show lines with different thicknesses to indicate to the user the relative size differences of the separate infusion line paths 721.

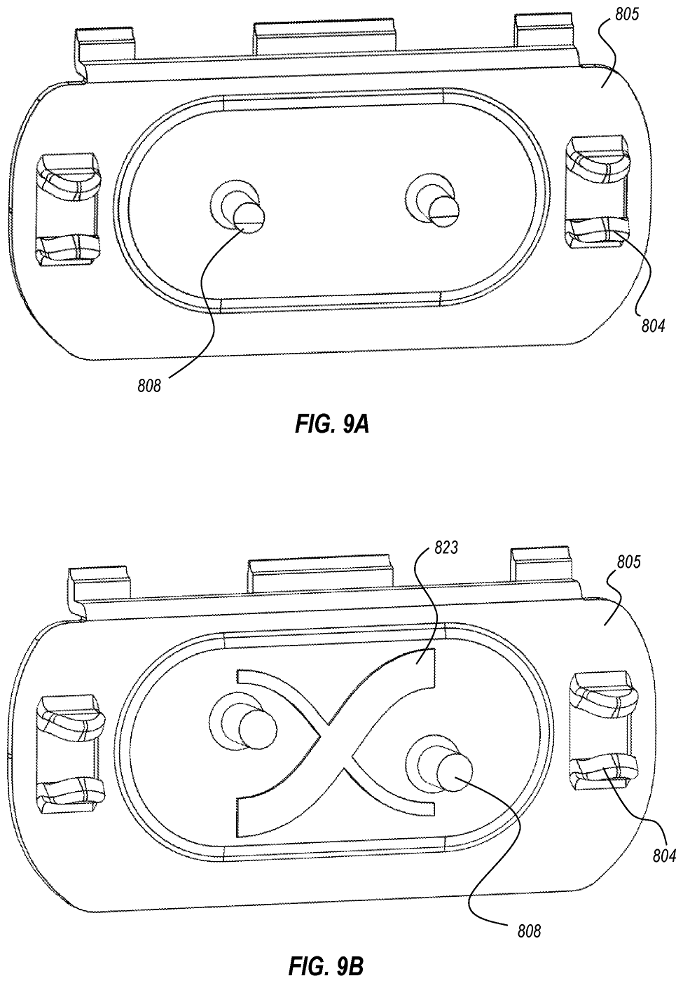

FIGS. 9A and 9B illustrate other embodiments of receiving surfaces 805 that include grip elements in the form of guide posts 808. The guide posts 808 can function as structures for guiding the infusion line in a manner that helps to secure the device to the infusion line. For example, in the FIG. 9A device, an infusion line can be routed through one set of grip elements 804, then “over” one of the guide posts 808, then “under” the next guide post, then through the last set of grip elements 804. The guide posts 808 can be included in addition to or as an alternative to centrally located grip elements of other embodiments.

The guide posts 808 can be substantially aligned with each other in the longitudinal direction, as shown in FIG. 9A. Alternatively, the guide posts 808 can be laterally offset from one another such that they are disposed on opposite sides of the longitudinal axis (as shown in FIG. 9B) and/or are different distances from the longitudinal axis. Such an offset can beneficially define differential paths with different levels of bending for the infusion line. For example, a larger infusion line may not require as much bending to be effectively secured, and could be impinged if bent excessively, whereas a smaller infusion line may require greater bending to be effectively secured.

In the illustrated example, a larger infusion line can be routed (from right to left) through a first set of grip elements 804, then over a first guide post 808, then under the next guide post 808, then through the second set of grip elements 804. A smaller infusion line can be routed (from right to left) through the first set of grip elements 804, then under the first guide post 808, then over the next guide post 808, then through the second set of grip elements 804. Because of the lateral offset of the guide posts 808, the smaller infusion line is forced into greater levels of bending, whereas the larger infusion line is also forced to bend but to a lesser degree.

The receiving surface 805 can also include one or more indicator markings 823 to aid the user in identifying how to route the infusion line through the device. For example, as shown, the indicator markings 823 can show lines with different thicknesses to indicate to the user preferred directions to route infusion lines based on relative infusion line size.

The example embodiments illustrated in FIGS. 9A and 9B each include two guide posts 808. While two guide posts 808 will typically best balance providing sufficient bending without overly impinging the infusion line, other embodiments may include more than two guide posts 808 (e.g., where relatively small infusion lines are expected). In some circumstances, a single guide post 808 may be sufficient to create effective bending and securement of the infusion line (e.g., where relatively large infusion lines are expected).

### Additional Terms & Definitions

As used herein, the “longitudinal axis” of an adaptor device is the axis that runs in the same general direction of an infusion line positioned therein, and will typically extend from one set of peripherally located grip elements on a first side of the device to an opposite set of peripherally located grip elements on a second side of the device (i.e., from “left” to “right” in the orientation of the Figures). The “lateral axis” is perpendicular to the longitudinal axis and extends along the width of the device (i.e., from “top” to “bottom” in the orientation of the Figures). A “longitudinal direction” or a “lateral direction” is any direction parallel to the longitudinal or lateral axis, respectively. The “horizontal plane” is the plane defined by the longitudinal and lateral axes, and can be, for example, parallel with the receiving surface of the device. The “vertical plane” is perpendicular to the horizontal plane (i.e., extends out of the page in the orientation of the Figures).

Unless indicated otherwise, as used herein, a “set of grip elements” includes at least a pair of opposed grip elements that are laterally spaced from one another a distance that allows an infusion line to be passed therebetween. Example embodiments illustrated herein include two or three sets of grip elements, though other embodiments may include a single set of grip elements or more than three sets of grip elements.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

Reference numerals referring to multiple instances of the same element may take the form of a reference numeral followed by a letter. For example, if component 150 is included multiple times in an embodiment, a general reference to such components and/or a reference to the collective set of such components may be referred to as “component 150,” whereas individual instances of the component may be referred to as “component 150a, component 150b, component 150c,” etcetera.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, the disclosed adaptor devices can optionally omit any non-disclosed grip element and/or other components (e.g., foam insert) intended to secure an infusion line or other medical line.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An adaptor device for attaching an indicator device to a medical line, the adaptor device comprising:
a longitudinal axis along which the medical line extends when the adaptor device is attached thereto;
a receiving surface; and
one or more sets of laterally opposing grip elements projecting from the receiving surface, each set of laterally opposing grip elements defining a space therebetween for receiving a medical line.

2. The device of claim 1, wherein at least one of the grip elements in each set of grip elements is a biased grip element capable of flexing to enlarge the space between the opposing grip elements to thereby accommodate medical lines of variable size.

3. The device of claim 1, further comprising a first section on which the receiving surface is disposed and a second section with which the first section is attached via a hinge.

4. The device of claim 3, wherein the first section and the second section define an inner compartment when in a closed configuration.

5. The device of claim 4, further comprising an indicator within the inner compartment.

6. The device of claim 5, wherein the indicator comprises a light and/or speaker.

7. The device of claim 3, further comprising a third section with which the second section is attached via a hinge.

8. The device of claim 7, wherein the second section and third section are closable to encompass the first section.

9. The device of claim 8, wherein the second section and third section define apertures through which the medical line is passable.

10. The device of claim 2, wherein the biased grip element includes a deformable arch structure that is biased toward an expanded state and is collapsible to increase the space between opposing grip elements.

11. The device of claim 10, wherein the arch structure includes a portion facing the opposing grip element, and wherein the portion facing the opposing grip element is not fixed to the receiving surface and is thereby free to move laterally toward and away from the opposing grip element.

12. The device of claim 1, wherein at least one of the grip elements includes fins biased toward a more lateral position and are capable of flexing toward a more longitudinal position to accommodate an inserted medical line.

13. The device of claim 1, wherein both of the grip elements in each set of grip elements are biased toward one another and capable of flexing to enlarge the space between the grip elements.

14. The device of claim 1, wherein at least one of the sets of grip elements is rotationally offset relative to the longitudinal axis.

15. The device of claim 1, wherein the grip elements of at least one of the sets of grip elements each include angled sections configured to force a bend in a medical line placed between the grip elements.

16. The device of claim 1, wherein the device comprises a set of grip elements that includes at least three laterally coincident grip elements arranged to define two or more medical line paths, wherein at least two of the two or more medical line paths are different in size to accommodate medical lines of different sizes.

17. The device of claim 1, wherein at least one of the sets of grip elements includes grip elements configured as guide posts for forcing a bend in a medical line.

18. The device of claim 1, wherein the device includes one or more sensors configured for sensing air bubbles, flow rate, precipitate formation, temperature, and/or salinity.

19. A medical line identification system, comprising:
two or more adaptor devices as in claim 1,
wherein the adaptor devices are configured to wirelessly communicate with one another such that when a first indicator associated with a first adaptor device is manually activated to provide an indicator signal, a second indicator associated with a second adaptor device is also automatically activated, without requiring manual activation, to provide a corresponding indicator signal.

20. A medical line sensor system, comprising one or more adaptor devices as in claim 1, each adaptor device comprising:
one or more sensors configured for sensing air bubbles, flow rate, precipitate formation, temperature, and/or salinity; and
one or more indicator components communicatively coupled to the one or more sensors and configured to activate according to predetermined sensor activity.
